(19) United States
(12) Patent Application Publication
(10) Pub. No.: US 2023/0134031 A1
(43) Pub. Date: May 4, 2023

(54) CONTROLLED PURGE RECOVERY USING TISSUE PLASMINOGEN ACTIVATOR (tPA)

(71) Applicant: ABIOMED, Inc., Danvers, MA (US)

(72) Inventors: Gary Stacey, Danvers, MA (US); Soumen Das, Danvers, MA (US); Scott C. Corbett, Danvers, MA (US); James Gabriel, Danvers, MA (US); Alexander Ship, Danvers, MA (US)

(73) Assignee: ABIOMED, Inc., Danvers, MA (US)

(21) Appl. No.: 17/975,929

(22) Filed: Oct. 28, 2022

Related U.S. Application Data
(60) Provisional application No. 63/273,424, filed on Oct. 29, 2021.

(51) Int. Cl. A61M 60/829; A61M 60/538; A61M 60/13; A61M 60/216; A61M 60/411

(52) U.S. Cl. CPC A61M 60/829; A61M 60/538; A61M 60/13; A61M 60/216; A61M 60/411

(57) ABSTRACT

A method for controlled purging of a blood pump and a blood pump assembly for implementing the method are provided. The blood pump is in fluid communication with a purging device. The purging device comprises a purge reservoir and a supplemental reservoir configured to be fluidically connected to the blood pump. The purge reservoir is configured to receive a purge fluid and the supplemental reservoir configured to receive a supplemental purge fluid. At least a portion of the blood pump is inserted into a patient and operated. A flow of the purge fluid is provided from the purge reservoir to the blood pump. A purge flow parameter at the blood pump is measured using a measuring device and when it is determined by a controller that the purge flow parameter meets a predetermined threshold, a remediation protocol is identified.

### CROSS-REFERENCE TO RELATED APPLICATION

This application claims priority from and the benefit of U.S. Provisional Application No. 63/273,424 filed Oct. 29, 2021, which is incorporated by reference herein in its entirety.

### TECHNICAL FIELD

This invention relates to a blood pump assembly comprising a blood pump, in particular an intravascular blood pump, to support a blood flow in a patient's blood vessel and methods for purging such a blood pump in operation while inserted into a patient.

### BACKGROUND

Blood pumps of different types are known, such as axial blood pumps, centrifugal blood pumps, or mixed-type blood pumps, where the blood flow is caused by both axial and radial forces. One example of a blood pump is the Impella® line of blood pumps (e.g., Impella 2.5®, Impella CP®, Impella 5.5®, etc.) which are products of Abiomed of Danvers, Mass.. Intravascular blood pumps are inserted into a patient's vessel such as the aorta by means of a catheter.

In some pump designs, a purge fluid is deployed to keep blood from entering the pump mechanism and to mitigate the effects of blood and the bio-deposit buildup on the pump mechanisms. For example, an anticoagulant such as heparin (typically the sodium salt of heparin) is used to preserve the patency of the pump components. The heparin is thought to keep the blood from coagulating in the gap between pump components such as an impeller shaft and the housing. Heparin is a commonly used anticoagulant typically administered in controlled dosages.

### BRIEF SUMMARY

Described herein are a method for controlled purging of a blood pump and a blood pump assembly for implementing the method. According to the described method, a blood pump that is in fluid communication with a purging device is provided. The purging device comprises a purge reservoir and a supplemental reservoir configured to be fluidically connected to the blood pump. The purge reservoir is configured to receive a purge fluid and the supplemental reservoir is configured to receive a supplemental purge fluid. At least a portion of the blood pump is inserted into a patient. The method also includes the steps of: operating the blood pump; providing a flow of the purge fluid from the purge reservoir to the blood pump; measuring, by a measuring device, a purge flow parameter at the blood pump; and identifying a remediation protocol upon determination, by a controller, that the purge flow parameter at the blood pump meets a predetermined threshold purge flow parameter.

Another aspect of the present disclosure relates to a blood pump assembly comprising a blood pump, a purging device in fluid communication with the blood pump, a measuring device configured to measure a purge flow parameter at the blood pump, and a controller. The purging device comprises a purge reservoir and a supplemental reservoir configured to be fluidically connected to the blood pump. The purge reservoir is configured to receive a purge fluid and the supplemental reservoir configured to receive a supplemental purge fluid. The controller is configured to monitor the purge flow parameter at the blood pump, and identify a remediation protocol upon determination that the purge flow parameter at the blood pump meets a predetermined threshold purge flow parameter.

In some embodiments, the blood pump comprises a motor section and a pump section, and the purge fluid and the supplemental purge fluid are supplied to the motor section.

The remediation protocol comprises: stopping the flow of the purge fluid from the purge reservoir to the blood pump, and starting a flow of the supplemental purge fluid from the supplemental reservoir to the blood pump; measuring, by the measuring device, a supplemental purge measurement at the blood pump; stopping the flow of the supplemental purge fluid from the supplemental reservoir to the blood pump upon determination, by the controller, that the supplemental purge flow parameter at the blood pump has fallen below or exceeds the predetermined threshold purge flow parameter indicating the supplemental purge fluid is no longer required; and resuming the flow of the purge fluid from the purge reservoir to the blood pump.

In some embodiments, the flow of the supplemental purge fluid is stopped and the flow of the purge fluid is resumed upon determining, by the controller, that the supplemental purge flow parameter at the blood pump exceeds the predetermined threshold purge flow parameter. In some embodiments, the flow of the supplemental purge fluid is stopped and the flow of the purge fluid is resumed upon determining, by the controller, that the supplemental purge flow parameter at the blood pump exceeds the predetermined threshold purge flow parameter by a predetermined amount, wherein the predetermined amount is at least 30% over a 24-hour period or at least 20% over a 12-hour period.

In other embodiments, the flow of the supplemental purge fluid is stopped and the flow of the purge fluid is resumed upon determining, by the controller, that the supplemental purge flow parameter at the blood pump has fallen below the predetermined threshold purge flow parameter. In some embodiments, the flow of the supplemental purge fluid is stopped and the flow of the purge fluid is resumed upon determining, by the controller, that the supplemental purge flow parameter at the blood pump has fallen below the predetermined threshold purge flow parameter by a predetermined amount, wherein the predetermined amount is at least 30% over a 24-hour period or at least 20% over a 12-hour period.

In some embodiments, the purge flow parameter is a purge flow rate and the predetermined threshold purge flow parameter is a decrease in the purge flow rate by a predetermined amount, wherein the predetermined amount is at least 30% within a 24-hour period or at least 20% over a 12-hour period. In some embodiments, the purge flow parameter is a purge flow rate and the predetermined threshold purge flow parameter is a decrease in the purge flow rate to below 3 mL/hour within a 6-hour period. In some embodiments, the purge flow parameter is a purge pressure and the predetermined threshold purge flow parameter is an increase in the purge pressure by a predetermined amount, wherein the predetermined amount is at least 30% within a 24-hour period or at least 20% over a 12-hour period. In some embodiments, the purge flow parameter is a purge pressure and the predetermined threshold purge flow parameter is an increase in the purge pressure to greater than 700 mmHg within a 24-hour period.

In some embodiments, the method further comprises deaerating the supplemental purge fluid prior to supplying the supplemental purge fluid to the blood pump.

In some embodiments, the supplemental purge fluid comprises tissue plasminogen activator (tPA). The supplemental purge fluid may also comprise dextrose. In some embodiments, the concentration of tPA is 2 mg/50 mL. The tPA may be lyophilized before it is combined with the purge fluid.

In some embodiments, the purge fluid comprises aqueous dextrose. For example, the purge fluid may comprise a solution of 5% dextrose in water. In some embodiments, the purge fluid may also comprise an anticoagulant, a pH controlling and buffering agent, or combination thereof. In some embodiments, the anticoagulant is warfarin, coumarin, heparin, or a direct thrombin inhibitor. In some embodiments, the direct thrombin inhibitor is lepirudin, desirudin, argatroban, bivalirudin, or mixtures thereof. In some embodiments, the pH controlling and buffering agent is sodium bicarbonate, sodium citrate, sodium lactate, sodium gluconate, sodium acetate, or sodium pyruvate.

### BRIEF DESCRIPTION OF DRAWINGS

Hereinafter, the invention will be explained by way of example with reference to the accompanying drawings. The accompanying drawings are not intended to be drawn to scale. For purposes of clarity, not every component may be labelled in every drawing. In the drawings:

FIG. 1 illustrates the blood flow and the purge flow through the gap between the shaft and the housing in the blood pump;

FIG. 2 is a schematic representation of an intravascular blood pump inserted before the left ventricle, with its inflow cannula positioned in the left ventricle;

FIG. 3 is a schematic longitudinal cross-section of an exemplary prior art blood pump;

FIG. 4 is an enlarged representation of a part of the blood pump of FIG. 3;

FIG. 5 is a schematic representation of a prior art blood pump assembly;

FIG. 6 is an embodiment of the purging cassette of the present invention; FIG. 7 is an embodiment of the purging cassette of the present invention during the implementation of a remediation protocol; and

FIG. 8 is a plot showing one example of a relationship between purge pressure and purge flow.

### DETAILED DESCRIPTION

Embodiments of the present disclosure are described in detail with reference to the drawing figures wherein like reference numerals identify similar or identical elements. It is to be understood that the disclosed embodiments are merely examples of the disclosure, which may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Blood pumps are deployed in patients that require critical and life-saving care. Consequently, it is important to remediate any aspect of the device that might adversely affect pump operation. Disclosed herein is an automation of one aspect of the operation of blood pump. In particular, the purge conditions are monitored by a controller and the need to replace the purge fluid with a supplemental purge fluid such as, purge fluid comprising tissue plasminogen activator (tPA), is detected.

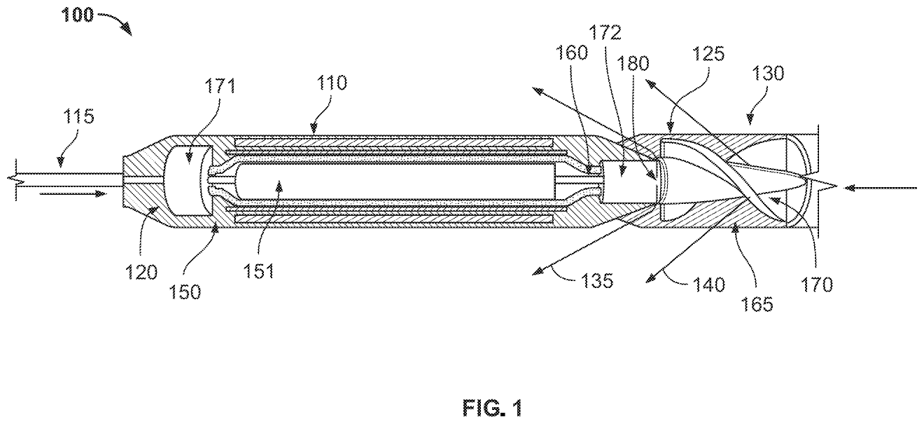

A blood pump of the aforementioned type is known, e.g., from EP 0 961 621 B1, which is incorporated by reference herein. With reference to FIG. 1, a pump 100 which possesses a drive section 110, a catheter 115 attached to the proximal end 120 of the drive section 110 (which is the end of the drive section closer to the clinician or “rear end” of the drive section) and having lines extending therethrough for the power supply to the drive section 110, and a pump section 130 fastened at the distal end 125 of the drive section. The drive section 110 comprises a motor housing 150 having an electric motor 151 disposed therein, with the motor shaft 160 of the electric motor distally protruding out of the drive section 110 and into the pump section 130. The pump section 130 in turn comprises a tubular pump housing 165 having an impeller 170 rotating therein which is seated on the end of the motor shaft 160 protruding out of the motor housing 150. The motor shaft 160 is mounted in the motor housing in two bearings 171, 172 which are maximally removed from each other in order to guarantee a true, exactly centered guidance of the impeller 170 within the pump housing 150. Different bearing types are used in different pump designs. As illustrated in FIG. 1, bearing 171 is a radial ball bearing and bearing 172 is an axial-radial sliding bearing. As illustrated in FIG. 1, blood 140 exits the outflow cage of the pump housing 165. Blood that would otherwise enter into the motor housing 150 is furthermore counteracted by a purge fluid 135 being passed through the motor housing and the impeller-side shaft seal bearing. Accordingly, the purge fluid passes through the gap of the impeller-side radial sliding bearing so as to prevent blood from entering into the housing. This is done at a purge pressure that is higher than the pressure present in the blood.

As illustrated in FIG. 1, the purge fluid 135 fills the motor housing 150 of the pump to form a lubricating film in the bearings 171, 172 of the pump. As described in US Patent Publication No. 20150051436, which is incorporated by reference herein, the purge fluid 135 can form a lubricating film in a bearing gap 180 of the axial slide bearing of a pump. Purge fluids are described as being fed through a purge fluid feed line and flowing through the radial bearing 171 located at the distal end of the motor housing 150 and then also flowing through the bearing gap 180 of the axial sliding bearing. The purge fluids fed in this manner are responsible for hemo-dilution and reduce blood retention time under the impeller 170.

To ensure that the purge fluid 135 reaches the distal radial bearing 172 at a pressure higher than the blood pressure present, there is provided, in at least one of the surfaces forming the bearing gap of the axial sliding bearing, a channel that penetrates the bearing gap 180 from radially outward to radially inward, so that the purge fluid can flow through this channel to the distal radial bearing. This channel need not necessarily lie in a bearing-gap surface, but can also be realized as a separate channel or as a bore. However, providing the channel in one of the bearing-gap surfaces has the advantage that the lubricating film in the bearing gap heats up less, because a part of the lubricating film is continually being replaced by purge fluid flowing in later. Preferably, the channel is located in the stationary bearing-gap surface in order to minimize the radial conveying capacity.

Typically, such purge fluids contain heparin. However, clinicians often do not want heparin to be administered to the patient's blood via the purge fluid. For instance, administration of heparin during any sort of surgical procedure may be counterproductive as it prevents the coagulation of blood and, thus, healing or hemostasis. Also, the amount of heparin administered to the patient's blood along with the purge fluid is difficult to control for various reasons. In particular, the amount of heparin is often more than what is desired by the clinicians, and the amount of heparin administered to the patient is difficult to precisely control. Accordingly, clinicians would often prefer to supply heparin to the patient separate from the operation of the blood pump, if needed (and then only in the amount needed). Furthermore, some patients are heparin-intolerant because they are susceptible to heparin-induced thrombocytopenia (HIT). So, a heparin-containing purge is not at all suitable for these patients. Accordingly, intravascular blood pumps are sometimes run, if desired, with a purge fluid that contains no or at least a reduced amount of heparin.

Another general problem arises with the heparin that is typically mixed into the purge fluid. Despite the purge fluid flowing through the gap formed between the shaft and the opening of the housing, thereby pushing back the blood which tends to enter the housing through such gap, blood ingress into the gap cannot entirely be prevented. In particular, some blood or blood components may always enter at least into a distal section of such gap. Heparin helps to prevent coagulation of the blood in the gap or adhesion of blood to the surfaces and, thus, prevents blockage of shaft rotation.

EP 3 542 837 A2, which is incorporated by reference herein, describes a pump that limits the use of a purge fluid, at least intermittently, to mitigate the consequences of the administration of heparin to a patient through the blood pump purge fluid. To accomplish this, EP 3 542 837 A2 proposes using a material for at least one surface of the sliding bearing having a relatively high thermal conductivity for the gap surfaces. Examples of such materials include silicon carbide. The opposing surface can be made of a ceramic material with a lower thermal conductivity (e.g., alumina toughened zirconia). As described, the shaft is made of alumina toughened zirconia and the sleeve in which the shaft is journaled is made of silicon carbide. Using special materials for pump components is therefore one solution that limits or even eliminates the use of a heparin-containing purge fluid.

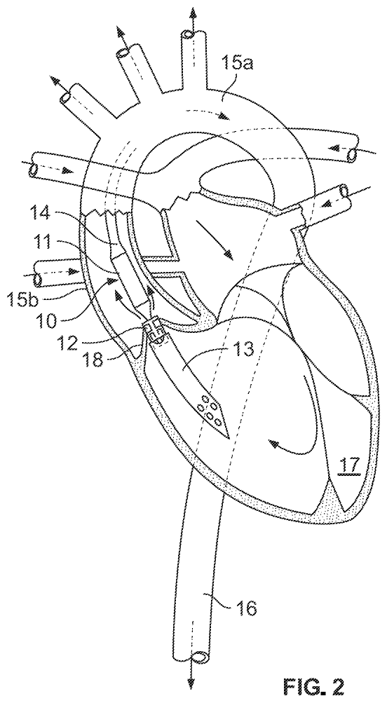

FIG. 2 represents the employment of a blood pump for supporting, in this particular example, the left ventricle. The blood pump comprises a catheter 14 and a pumping device 10 attached to the catheter 14. The pumping device 10 has a motor section 11 and a pump section 12 which are disposed coaxially one behind the other and result in a rod-shaped construction form. The pump section 12 has an extension in the form of a flexible suction hose 13, often referred to as “cannula.” An impeller is provided in the pump section 12 to cause blood flow from a blood flow inlet to a blood flow outlet, and rotation of the impeller is caused by an electric motor disposed in the motor section 11. The blood pump is placed such that it lies primarily in the ascending aorta 15b. The aortic valve 18 comes to lie, in the closed state, against the outer side of the pump section 12 or its suction hose 13, which, when the pump is positioned, is proximate the left ventricle 17. The blood pump with the suction hose 13 in front is advanced into the represented position by advancing the catheter 14, optionally employing a guide wire. In so doing, the suction hose 13 passes the aortic valve 18 retrograde, so the blood is sucked in through the suction hose 13 and pumped into the aorta 16 via the aortic arch 15a.

The use of the blood pump is not restricted to the application represented in FIG. 2, which merely involves a typical example of application. Thus, the pump can also be inserted through other peripheral vessels, such as the subclavian artery. Alternatively, reverse applications for the right ventricle may be envisioned.

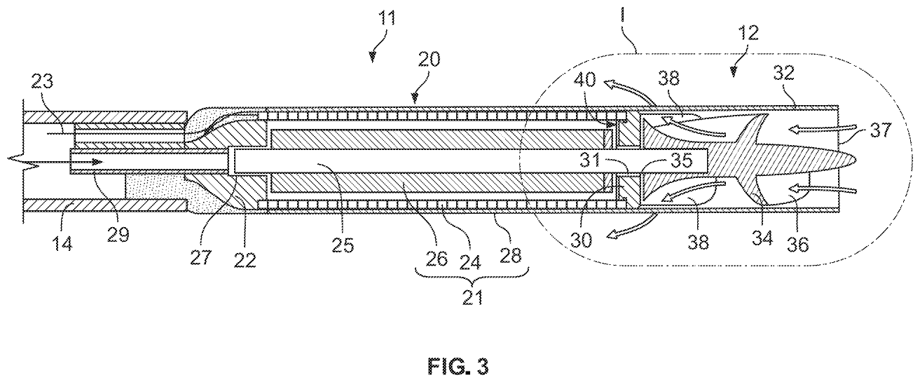

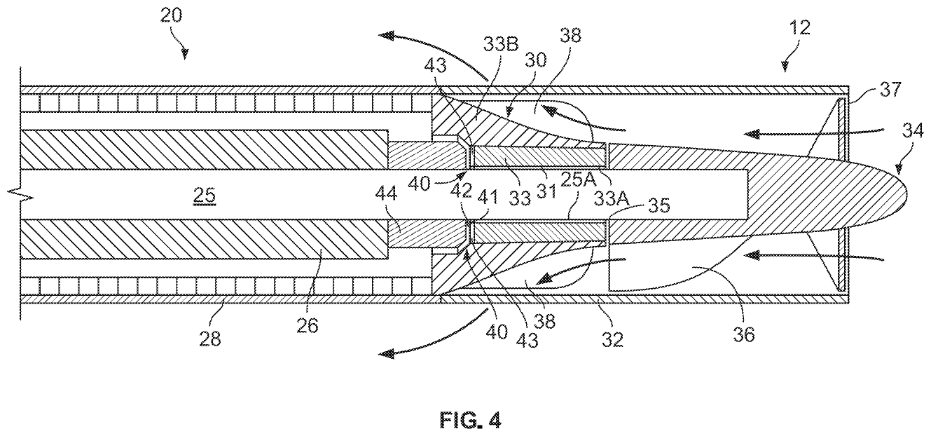

FIG. 3 shows an exemplary embodiment of the blood pump as described in U.S. Patent Application Publication No. 2015/0051436 A1, which is incorporated by reference herein, and which is likewise suitable for use in the context of the present invention, except that the encircled front end marked with “I” may be modified, such modification being shown in FIG. 4. Accordingly, the motor section 11 has an elongated housing 20 in which an electric motor 21 may be housed. A stator 24 of the electric motor 21 may have, in the usual way, numerous circumferentially distributed windings as well as a magnetic return path 28 in the longitudinal direction. The magnetic return path 28 may form an outer cylindrical sleeve of the elongate housing 20. The stator 24 may surround a rotor 26 connected to the motor shaft 25 and consisting of permanent magnets magnetized in the active direction. The motor shaft 25 may extend over the entire length of the motor housing 20 and protrude distally out of the latter through an opening 35. There, it carries an impeller 34 with pump vanes 36 projecting therefrom, which may rotate within a tubular pump housing 32 which may be firmly connected to the motor housing 20.

The proximal end of the motor housing 20 has the flexible catheter 14 sealingly attached thereto. Through the catheter 14, there may extend electrical cables 23 for power supply to and control of the electric motor 21. In addition, a purge fluid line 29 may extend through the catheter 14 and penetrate a proximal end wall 22 of the motor housing 20. Purge fluid may be fed through the purge fluid line 29 into the interior of the motor housing 20 and exit through the end wall 30 at the distal end of the motor housing 20. The purging pressure is chosen such that it is higher than the blood pressure present, in order to thereby prevent blood from penetrating into the motor housing, being between 300 and 1400 mmHg depending on the case of application.

As mentioned before, the same purged seal can be combined with a pump that is driven by a flexible drive shaft and a remote motor.

Upon a rotation of the impeller 34, blood is sucked in through the distal opening 37 of the pump housing 32 and conveyed backward within the pump housing 32 in the axial direction. Through radial outlet openings 38 in the pump housing 32, the blood flows out of the pump section 12 and further along the motor housing 20. This ensures that the heat produced in the motor is carried off. It is also possible to operate the pump section with the reverse conveying direction, with blood being sucked in along the motor housing 20 and exiting from the distal opening 37 of the pump housing 32.

The motor shaft 25 is mounted in radial bearings 27, 31 at the proximal end of the motor housing 20, on the one hand, and at the distal end of the motor housing 20, on the other hand. The radial bearings, in particular the radial bearing 31 in the opening 35 at the distal end of the motor housing, are configured as sliding bearings. Furthermore, the motor shaft 25 is also mounted axially in the motor housing 20, the axial bearing 40 likewise being configured as a sliding bearing. The axial sliding bearing 40 serves for taking up axial forces of the motor shaft 25 which act in the distal direction when the impeller 34 conveys blood from distal to proximal. Should the blood pump be used for conveying blood also or only in the reverse direction, a corresponding axial sliding bearing 40 may (also or only) be provided at the proximal end of the motor housing 20 in a corresponding manner.

FIG. 4 shows the portion marked with “I” in FIG. 3 in greater detail, yet structurally modified. There can be seen in particular the radial sliding bearing 31 and the axial sliding bearing 40. The bearing gap of the radial sliding bearing 31 is formed, on the one hand, by the circumferential surface 25A of the motor shaft 25 and, on the other hand, by the surface 33A of a through bore in a bushing or sleeve 33 of the motor housing's 20 end wall 30 defining an outer gap diameter of about 1 mm, but the outer gap diameter may also be larger than this. In one example, the bearing gap of the radial sliding bearing 31 has a gap width of 2 μm or less not only at the front end or impeller-side of the gap but over the entire length thereof. Preferably the gap width is between 1 μm and 2 μm. The length of the bearing gap may range from 1 mm to 2 mm, preferably from 1.3 mm to 1.7 mm, e.g., 1.5 mm. The surfaces forming the gap of the radial sliding bearing 31 have a surface roughness of 0.1 μm or less. These dimensions will vary with the type of pump and are presented by way of example and not by way of limitation.

The bearing gap of the axial sliding bearing 40 is formed, on the one hand, by the axially interior surface 41 of the end wall 30 and a surface 42 opposing it. This opposing surface 42 is part of a ceramic disk 44 which is seated on the motor shaft 25 distally of the rotor 26 and rotates with the rotor 26. A channel 43 in the bearing gap surface 41 of the end wall 30 ensures that purge fluid can flow through between the bearing gap surfaces 41 and 42 of the axial sliding bearing 40 to the radial sliding bearing 31 and exit from the motor housing 20 distally. The axial sliding bearing 40 represented in FIG. 3 is a normal sliding bearing. Unlike the representation, the axial gap of the axial sliding bearing 40 is very small, being a few

Instead of the axial sliding bearing 40 and radial sliding bearing 31, there can also be realized a combined radial-axial sliding bearing 40 having a concave bearing shell in which a convex bearing surface runs. Such a variant is represented in FIG. 4 by a spherical sliding bearing 40. The bearing-gap surface 41 is of spherically concave design, and the opposing bearing-gap surface 42 is of corresponding spherically convex design. The channel 43 again lies in the stationary bearing-gap surface 41 of the end wall 30. Alternatively, the stationary bearing-gap surface 41 of the end wall 30 can be of convex configuration and the opposing bearing-gap surface 42 of concave configuration. The surfaces 42, 43 can also be conical instead of spherical. Preferably, a corresponding radial-axial sliding bearing is provided on both sides of the motor housing 20 in order not to permit any radial offset upon axial travel of the shaft 25. The advantage of a combined axial-radial sliding bearing lies in the higher loading capacity. However, a disadvantage is the greater frictional diameter.

In some embodiments, the blood pump is inserted into a blood vessel of the patient through a sheath. In some embodiments, no sheath is used. In other embodiments, the blood pump is inserted using a guidewire.

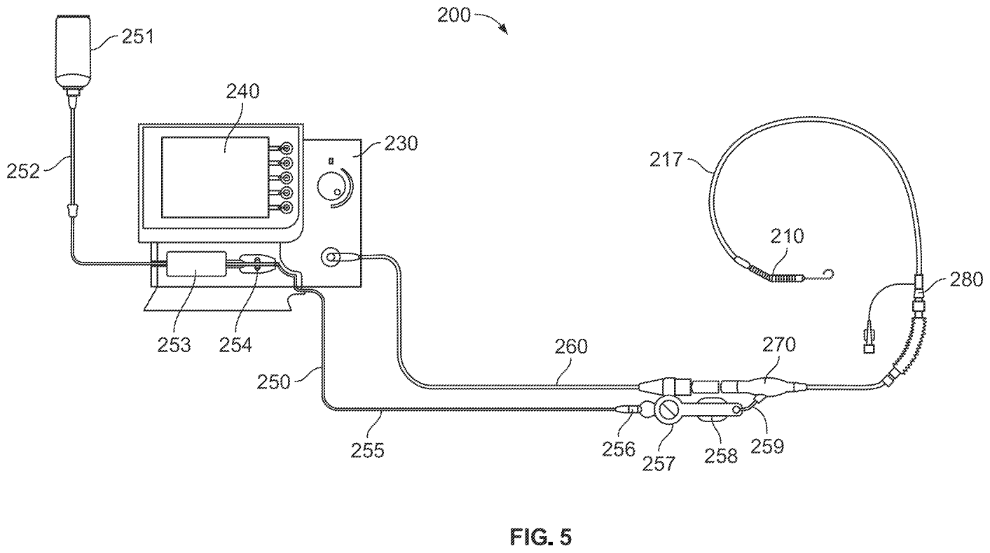

As shown in FIG. 5, a blood pump assembly 200 may comprise a blood pump 210 fluidically connected to a purging device 250. The blood pump assembly 200 also includes a controller 230 (e.g., an Automated Impella Controller® from Abiomed, Inc., Danvers, Mass.), a display 240, a connector cable 260, a plug 270, and a repositioning unit 280. As shown, controller 230 includes display 240. Controller 230 monitors and controls blood pump 210. During operation, purging device 250 delivers a purge fluid to blood pump 210 through catheter tube 217 to prevent blood from entering the motor (not shown) within motor housing 216. In some implementations, the purge fluid comprises a dextrose solution (e.g., 5% dextrose in water with 25 or 50 IU/mL of heparin). Connector cable 260 provides an electrical connection between blood pump 210 and controller 230. Plug 270 connects catheter tube 217, purging device 250, and connector cable 260. In some embodiments, plug 270 includes a memory for storing operating parameters in case the patient needs to be transferred to another controller. Repositioning unit 280 may be used to reposition blood pump 210.

As shown, purging device 250 comprises a reservoir 251, purge fluid supply line 252, a purge cassette 253, a purge disc 254, purge tubing 255, a check valve 256, a pressure reservoir 257, an infusion filter 258, and a sidearm 259. Reservoir 251 may, for example, be a bag or a bottle. A purge fluid is stored in reservoir 251. A purge fluid spike at the end of purge fluid supply line 252 may be used to puncture reservoir 251 and connect the purge fluid in reservoir 251 to purge fluid supply line 252. Purge fluid supply line 252 carries the purge fluid from reservoir 251 to purge cassette 253. Purge tubing 255 carries the purge fluid from purge cassette 253 to blood pump 210.

Purge cassette 253 controls how the purge fluid in reservoir 251 is delivered to blood pump 210 and the flow path of the purge fluid from reservoir 251 to blood pump 210. For example, purge cassette 253 may include one or more valves (e.g., purge path diverters) for controlling a pressure and/or flow rate of the purge fluid. In addition to containing the components for delivering the purge fluid, purge cassette 253 also maintains the pressure barrier between the blood and the motor of blood pump 210 to prevent blood from entering the motor. Purge cassette 253 may contain a rack and pinion which is attached to a piston. Purge disc 254 includes one or more measuring device, such as pressure sensors (e.g., a pressure-sensing diaphragm) for measuring purge pressure of the purge fluid at blood pump 210. Controller 230 is connected to purge cassette 253 and purge disc 254. Purge disc 254 transmits pressure to controller 230 based on the purge pressure in purge tubing 255. A sensor in controller 230 measures the pressure so that it can be displayed on screen 240. Controller 230 may include a stepper motor. A tic (or step) represents stepper motor positions in units of microsteps, which are also called pulses. In some embodiments, a purge pressure/purge flow curve algorithm is deployed by the controller 230 along with the number of steps/minute of the stepper motor, and pressure measurement by purge disc 254 to calculate the corresponding purge flow rate.

As described above, purge tubing 255 provides a fluidic connection passing through purge cassette 253 to blood pump 210. In some embodiments, a Y-connector and/or a yellow Luer connector are also provided, which facilitate a continuous fluid path or purge fluid path. Y-connector is an adapter that connects purge tubing 255 to blood pump 210. Yellow Luer connector connects purge tubing 255 to a check valve (yellow luer lock) on blood pump 210. Pressure reservoir 257 provides additional filling volume during a purge fluid change. In some embodiments, pressure reservoir 257 includes a flexible rubber diaphragm that provides the additional filling volume by means of an expansion chamber. Infusion filter 258 helps prevent bacterial contamination and air from entering catheter tube 217. Sidearm 259 provides a fluidic connection between infusion filter 258 and plug 270.

During operation, controller 230 receives measurements from purge disc 254 and controls the stepper motor's number of ticks to control the purge pressure. In some embodiments, during operation, purge cassette 253 is placed in controller 230 and connected with blood pump 210. As noted above, controller 230 controls and measures purge pressure and calculates purge flow rate via purge cassette 253 and/or purge disc 254. Controller 230 may also control the purge fluid supply. During operation, after exiting purging device 250 through sidearm 259, the purge fluid is channeled through purge lumens (not shown) within catheter tube 217 and plug 270. Sensor cables (not shown) within catheter tube 217, connector cable 260, and plug 270 provide an electrical connection between purge disc 254 and controller 230. Motor cables (not shown) within catheter tube 217, connector cable 260, and plug 270 provide an electrical connection between the motor within motor housing 216 and controller 230. During operation, controller 230 receives measurements from purge disc 254 through the sensor cables and controls the electrical power delivered to the motor within motor housing 216 through the motor cables. By controlling the power delivered to the motor within motor housing 216, controller 230 can control the speed of the motor within motor housing 216. In some embodiments, controller 230 includes safety features to prevent air from entering purge tubing 255. Controller 230 may include circuitry for monitoring the motor current for drops in current indicating air in the line. Controller 230 may include warning sounds, lights or indicators to alert an operator of disconnects or breaks in purge tubing 255 which may result in the introduction of air to the line.

Various modifications can be made to blood pump assembly 200 and one or more of its components. For example, as detailed in Abiomed, Impella® Ventricular Support Systems for Use During Cardiogenic Shock and High-Risk PCI: Instructions for Use and Clinical Reference Manual, Document No. 0042-9028 rG (April 2020), which is incorporated herein by reference, blood pump assembly 200 can be modified to accommodate other types of blood pumps, such as the Impella 2.5®, Impella LD®, and Impella CP® catheters. As another example, one or more additional measuring devices may be added to blood pump 210. For example, as described in U.S. Patent Application Publication No. 2020/0288988 A1, which is incorporated herein by reference, a signal generator may be added to blood pump 210 to generate a signal indicative of the rotational speed of the motor within motor housing 216. As another example, a second measuring device which is a pressure sensor may be added to blood pump 210 near inlet area 212 that is configured to measure a left ventricular blood pressure. In such embodiments, additional sensor cables may be disposed within catheter tube 217, connector cable 260, and plug 270 to provide an electrical connection between the one or more additional measuring devices and controller 230. As yet another example, one or more components of blood pump assembly 200 may be separated. For example, display 240 may be incorporated into another device in communication with controller 230 (e.g., wirelessly or through one or more electrical cables).

Display 240 can provide useful information to the user of the blood pump assembly 200. For example, U.S. Patent Application Publication No. 2020/0376183A1, which is incorporated herein by reference, describes and illustrates some of the types of information that it can display, which may relate not only to the characteristics of the blood pump assembly (e.g., blood pump type, serial number, software version, etc.), but also to the operation of the blood pump assembly (e.g., present blood pump speed (performance) setting, blood pump flow measurements, purging device measurements, a status indicator, etc.). Some of this information may be obtained from purge disc 254 described above. Display 240 can also provide notifications to the user. For example, a notification may serve as an alert and include a statement describing the cause of the alert. In some embodiments, display 240 may be a touchscreen and a user may switch between screens by tapping button labels on display 240. In some embodiments, a user may use a separate input device, such as a mouse or a keyboard, to switch between screens.

A system for monitoring and/or controlling a plurality of medical device controllers, such as controller 230, is described and illustrated in U.S. Patent Application Publication No. 2020/0376183A1. System may include medical device controllers, computer network, local area network (LAN), remote link module, router, wireless access point, cell site, server, data store, OCR engine, and/or monitoring stations. Computer network may include wired and/or wireless segments and/or networks. The medical device controllers can be connected to computer network using a variety of known techniques. For example, the medical device controllers can be directly connected to computer network, through remote link module, or through LAN, router, and wireless access point. Server may be configured to request status information from medical device controllers through computer network. In some embodiments, server requests the status information automatically and/or repeatedly. Server may also be configured to process the received status information.

Data store may be configured to store unprocessed and/or processed status information. Data store may also be configured to provide at least some of the unprocessed and/or processed status information to monitoring stations upon request. Monitoring stations may be, for example, a phone, tablet, and/or computer. In some embodiments, monitoring stations may use cloud-based technology to securely and remotely display at least some of the unprocessed and/or processed status information on associated displays. For example, monitoring stations may use an online device management system, such as the Impella Connect® from Abiomed, Inc., Danvers, Mass., to securely and remotely display at least some of the unprocessed and/or processed status information. In some embodiments, server and/or monitoring stations may also be configured to remotely send commands to one or more medical device controllers within system. In some embodiments, one or more medical device controllers within system may offload one or more computations to server and/or monitoring stations. For example, if controller 230 is added to the system, controller 230 may offload complex calculations (e.g., machine learning algorithms) to server and/or monitoring stations. To reduce latency, controller 230 may also offload such calculations to another computing device on the same LAN.

During operation, the blood pump is attached to a purge fluid source (such as the purge fluid reservoir), and fluid passes into the motor housing through the purge fluid line. The purge fluid then flows through the axial sliding bearing and further through the distal radial bearing. In the axial sliding bearing the purge fluid forms the lubricating film in the bearing gap. The pressure at which the purge fluid flows through the motor housing has an adverse effect, however, on the width of the bearing gap. Specifically, higher purge pressure requires a smaller bearing gap width which results in a thinner lubricating film between the sliding surfaces. The thinner the lubricating film is, the greater the motor current that is required for driving the electric motor to overcome the frictional forces. This complicates the control of the blood pump, because the current conveying volume is normally established by stored characteristic curves solely on the basis of the motor current and the rotational speed (both known quantities). When the purge pressure also affects the motor current, this factor also has to be taken into consideration. In view of the fact that the same blood pump type can be operated for a great variety of applications with different purge pressures between 300 mmHg and 1400 mmHg, it is important to avoid a dependence of motor current on purge pressure.

Such dependence is avoided when a purge fluid having a viscosity that is considerably higher than the viscosity of water (η=0.75 mPas at 37° C.) is selected. In purge fluids with dextrose, the viscosity of the purge fluid is controlled by the concentration of dextrose in the purge fluid. Aqueous solutions of dextrose are widely administered to patients for a variety of reasons. The amount of dextrose in the aqueous solution is about 5% to about 50%. In one embodiment, the purge fluid contains 5% dextrose in water (i.e., 278 mmol/liter). The viscosity can be increased by including solutions with a higher concentration of dextrose in water (e.g., D20W, D40W, etc.). When a highly viscous purge fluid is used, the fluid film is maintained even at high pressures and the friction of the axial sliding bearing is accordingly independent of the purge pressure. In some embodiments, the axial sliding bearing can be configured as a simple sliding bearing, and does not have to be configured as a hydrodynamic sliding bearing, when a purge fluid having a viscosity at 37° C. that is about 1.2 mPas or higher. Therefore, when purge fluids that contain no or less heparin are considered, the viscosity of such purge fluids still needs to be considered.

The pump impeller does induce shear stress on the blood passing through the pump. Shear stress is induced predominantly in the gap between the impeller and the outer face of the ceramic bearing and between the impeller shaft and the inner race of the bearing (e.g., ceramic bearings, ball bearings, etc.). Due to the shear stresses to which the blood is subjected, blood proteins denature and polymerize as the blood passes through the pump. The deposition of the denatured and agglomerated protein causes activation of the clotting cascade, which, in turn, causes the buildup of bio-deposits on the pump mechanisms (e.g., the impeller, the outflow cage, etc.). The bio-deposit buildup will cause the motor current needed to operate the pump to increase. The increased motor current or bio-deposits can degrade pump performance or even cause a pump stop.

As noted above, to mitigate the adverse effects of shear on the blood that flows through the pump or to mitigate the bio-deposit buildup caused by the collection of blood debris in the purge fluid pathway, the purge fluids used in purged blood pumps typically include the anticoagulant heparin (e.g., 50 units/mL) in 5%-Dextrose (D5W). The dextrose concentration determines the viscosity of the purge fluid and hence affects the purge flow rate. Purge fluids with lower dextrose concentrations are less viscous and flow more quickly with less pressure through the purge system. Purge fluids with higher dextrose concentrations (more viscous) result in a lower purge flow rate and require a greater purge pressure. A reduction in dextrose concentration from 20% to 5% results in an approximately 30% to 40% increase in purge flow rates.

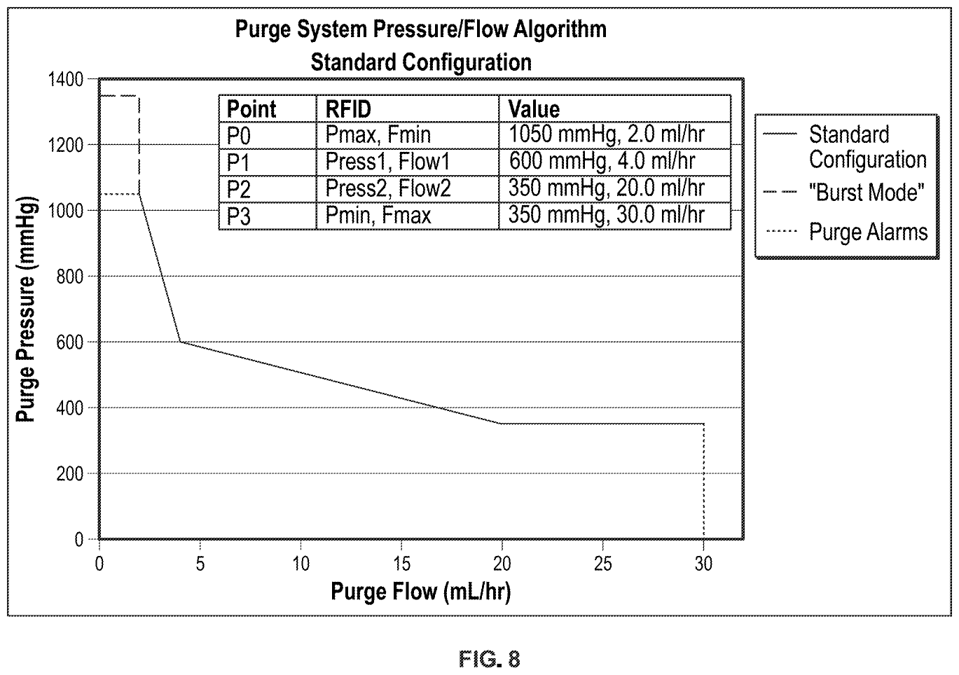

Purge flow rates are typically in the range of about 2 mL/hour to about 30 mL/hour as illustrated in FIG. 8. This results in a purge pressure of about 1,000 mmHg to about 300 mmHg. Typical purge flow rates for the blood pumps described herein, e.g., Impella CP®, Impella 2.5®, Impella 5.0®, Impella LD®, and Impella RP®, are about 5 mL/hour to about 20 mL/hour. These pumps all have a ball-bearing rotor/stator system with similar tolerances leading to similar purge operation ranges. Typical purge flows for the Impella 5.5° are about 2 mL/hour to about 10 mL/hour. This lower flow rate results from the deployment of a ceramic bearing rotor/stator system designed with a reduced purge gap (radial) to reduce or eliminate the amount of heparin delivered to the patient. For surgical patients, surgeons prefer not to administer heparin in the first few days after surgery. For these patients, then, purge fluids that contain no heparin are preferred.

Consistent purge flow is used to keep two important regions clear of debris: 1) the gap between the rotor shaft and sleeve bearing; and 2) the gap between the sleeve bearing and the impeller. Due to diffusion and flow co-mixing, some blood components may potentially reach these gaps. Heparin in the purge solution enhances protection against ingress, adsorption, deposition, and coagulation of blood components. It also improves the working life of the bearings, for at least the reasons stated below.

Specifically, continuous and dynamic physical adsorption (physisorption) of heparin onto the surfaces around the purge path reduces adsorption of blood components and, thus, prevents bio-deposition of blood debris on the bearings and other pump components. Also, heparin partially neutralizes the slightly acidic D5W solution, which helps to maintain the physiological pH in the aforementioned gaps and, therefore, reduces the risk of blood protein denaturing. Locally elevated concentration of heparin, both under the impeller and inside the sleeve bearing gap, may also reduce the risk of blood coagulation in these areas. Addition of heparin increases the electric conductivity of the purge fluid and therefore reduces the negative impact of electrostatic discharge on the bearing working life.

Therefore, heparin is provided in the purge fluid to prevent the formation of shear-induced bio-material or bio-deposits, and the resulting undesirable deposition/accumulation of biological material in the pump, such as between the impeller shaft and the inner race of the bearings at high shear areas. However, as noted above, there are challenges associated with adding heparin to the purge fluid. Specifically, heparin: a) makes systematic anticoagulant management complex (i.e., there is a need to consider the heparin dose that the patient is receiving via the purge fluid); b) heparin, as an anticoagulant, increases a patient's propensity to bleed; c) heparin makes it more difficult to control bleeding in post-operative patients, especially when surgical devices are used on such patients; and d) heparin cannot be used for heparin-induced thrombocytopenia (HIT) patients.

To mitigate problems in the pump performance when the purge fluid has no or low concentration of heparin, the use of a different purge fluid/purge fluid additive has been previously proposed. For example, U.S. Provisional Application No. 63/017,445, filed Apr. 29, 2020, which is incorporated by reference herein, proposes using a purge fluid comprising a pH controlling and buffering agent. Non-limiting examples of a suitable pH controlling and buffering agent include, for example, sodium bicarbonate, sodium citrate, sodium lactate, sodium gluconate, sodium acetate, or sodium pyruvate. In some embodiments, the pH controlling and buffering agent is sodium bicarbonate. In one example, the pH of sodium bicarbonate is about 7.4 to about 9.1. Other ranges, include but are not limited to about 7.5 to about 9.1, 7.6 to about 9.1, 7.7 to about 9.1, 7.8 to about 9.1, 7.9 to about 9.1, 8.0 to about 9.1, 8.1 to about 9.1, 8.2 to about 9.1, 8.3 to about 9.1, 8.4 to about 9.1, 8.5 to about 9.1, 8.6 to about 9.1, 8.7 to about 9.1, 8.8 to about 9.1, 8.9 to about 9.1 and 9.0 to about 9.1.

The pH controlling and buffering agents are added to a purge fluid containing dextrose alone or in combination with a reduced amount of heparin. The concentration of the pH-controlling and buffering agent in a solution with aqueous dextrose is selected to provide a solution with a pH within the range prescribed above. In one embodiment, a solution containing bicarbonate is mixed with a dextrose solution, such as dextrose 5% in water (D5W), dextrose 20% in water (D20W) dextrose 40% in water (D40W), etc. The amount of bicarbonate in the solution of bicarbonate mixed with the dextrose solution is about 1.5 milliequivalents per liter (mEq/L) to about 50 mEq/L. In some embodiments, the purge fluid solution may contain a reduced amount of heparin along with the pH controlling and buffering agents described above. In some embodiments, reduced concentrations of about 12.5 units/mL of heparin or less are contemplated. In some embodiments, reduced concentrations of about 6.25 units/mL or less are also contemplated. In some embodiments, reduced concentrations in the range of about 1 unit per mL to about 6.25 units/mL are also contemplated.

If a patient is intolerant to heparin, due to heparin-induced thrombocytopenia (HIT), but there is still a need to add an anticoagulant to the purge solution of pH controlling and buffering agent combined with aqueous dextrose, a direct thrombin inhibitor (DTI) can be added to the solution. If a DTI is added to the purge solution, the concentration of the DTI in the purge solution should be a dose equivalent of about 0.01 mg/kg/hour. to about 0.012 mg/kg/hour. The dose equivalent is selected to provide a partial thromboplastin test (PTT) time of about 40-50 seconds. Non-limiting examples of a suitable DTI include, for example, lepirudin, desirudin, argatroban, bivalirudin, or mixtures thereof. The concentration of the DTI in the purge solution is about 20 mg/500 mL to about 60 mg/500 mL.

Another solution that has been previously proposed to mitigate problems in the pump performance due to the undesirable deposition/accumulation of biological material in the pump involves the use of a supplemental purge fluid comprising tissue plasminogen activator (tPA). See, e.g., Sorensen, E. N. et al., “Use of Tissue Plasminogen Activator to Resolve High Purge System Pressure in a Catheter-based Ventricular-assist Device,” The J. of Heart & Lung Transplantation, 33(4): 457-8 (April 2014) (hereafter “Sorensen”); Oetken H. et al., “Use of Tissue Plasminogen Activator Via Purge System in a Catheter-based Ventricular Assist Device,” Proceedings of the 48th Critical Care Congress, San Diego, Calif. (Feb. 17-20, 2019), Critical Care Medicine, Abstract No. 159 (January 2019) (hereafter “Oetken”). tPA is a protein involved in the breakdown of blood clots. It is a serine protease found on endothelial cells, the cells that line the blood vessels. As an enzyme, it catalyzes the conversion of plasminogen to plasmin, the major enzyme responsible for clot breakdown. Sorensen discloses that for a patient with a blood pump inserted into the left ventricle, when the purge pressure increased to >700 mmHg, 2 mg tPA diluted in 50 mL normal saline was timely substituted for heparinized purge solution. This helped in the resolution of the high purge pressure. Oetken discloses that for a patient with a blood pump inserted into the heart, when the purge pressure increased to >900 mmHg and the purge flow rate decreased to 1-3 mL/hour, 4 mg tPA diluted in 100 mL of dextrose 5% sterile water was timely substituted for heparinized purge solution. This helped in the resolution of the high purge pressure and low purge flow rate. The Sorensen authors believe that tPA initiates local fibrinolysis by binding to fibrin in a clot and converting plasminogen to plasmin. The alternative to the timely use of tPA in Sorensen and Oetken would have been an emergency operation to exchange the inserted blood pump with a new one.

Sorensen and Oetken demonstrate that when there is a decrease in purge flow rate or an increase in purge pressure compared to normal values due to buildup of biological material in the pump, tPA administration can help resolve the issue. However, tPA has to be administered in a timely manner for it to be effective. Currently, the blood pump user has to manually monitor the purge flow rate and/or purge pressure and when it appears to be abnormal, the user has to make a judgement call as to when to administer tPA. However, the challenge is in determining when to deliver the tPA. A delayed administration of tPA when purge pressure is too high reduces the chance of purge recovery due to insufficient concentration of tPA delivered at the site of biological material buildup or clot. The current procedure of the user first obtaining tPA and then replacing the purge fluid with purge fluid comprising tPA can also lead to unwanted delays in tPA administration.

The present disclosure provides a method and a blood pump assembly to enable a controlled purge recovery in a timely and convenient manner. One aspect of the present disclosure relates to an automated process to monitor certain purge flow parameters that could be indicative of potential mechanical issues with the pump motor due to biomaterial buildup and timely identify the administration of tPA to maximize the chance of purge recovery. Another aspect of the present disclosure relates to a blood pump assembly to conveniently administer tPA without delay.

The blood pump assembly 300 may comprise a blood pump 210 fluidically connected to a purging device 350. The blood pump assembly 300 also includes a controller 230 (e.g., an Automated Impella Controller® from Abiomed, Inc., Danvers, Mass.), a display 240, a connector cable 260, a plug 270, and a repositioning unit 280, all of which may be substituted for the blood pump assembly 200 illustrated in FIG. 5.

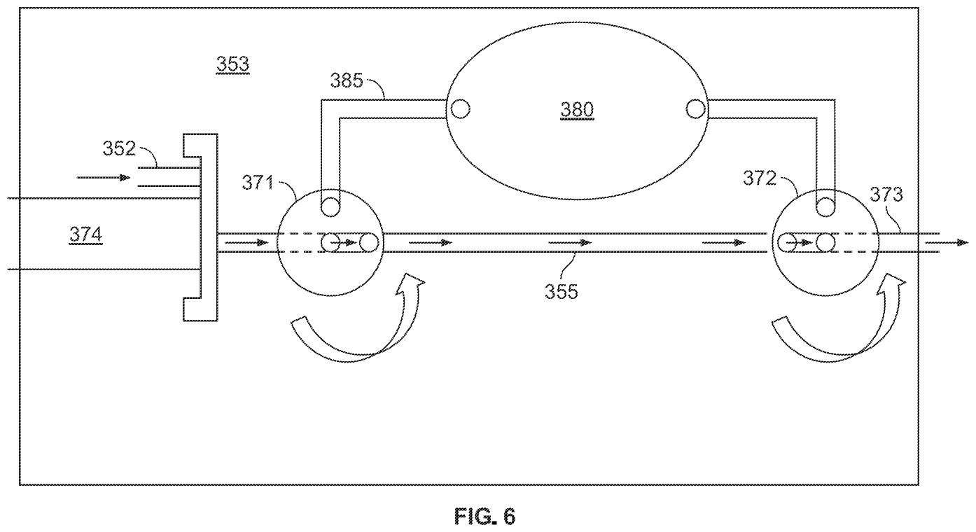

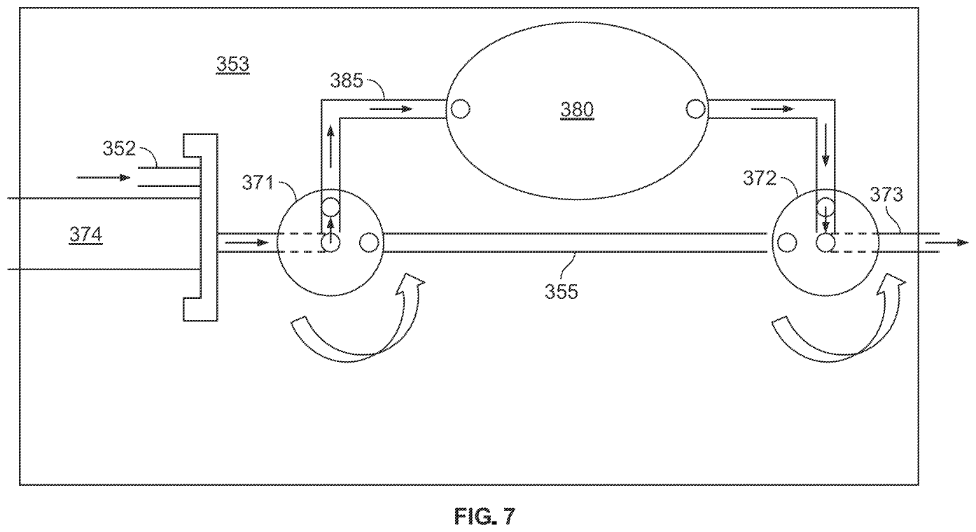

Purging device 350 comprises a purge fluid reservoir 351, purge fluid supply line 352, a purge cassette 353, a purge disc 354, purge tubing 355, a check valve 356, all of which may be substituted for purging device 250 illustrated in FIG. 5. FIGS. 6 and 7 show an exemplary purge cassette 353 of an embodiment of the present disclosure. However, purge cassette 353 also comprises a supplemental purge fluid reservoir 380, a supplemental purge fluid supply line 385, a first purge path diverter 371, and a second purge path diverter 372. Reservoir 351 (similar to reservoir 251 in FIG. 5) may, for example, be a bag or a bottle. A purge fluid is stored in reservoir 251. Purge fluid supply line 352 provides a fluidic connection between reservoir 351 and purge cassette 353. In some embodiments, a piston and cylinder 374 is used to drive the flow of the purge fluid from reservoir 351 through purge fluid supply line 352 to purge cassette 353. Purge cassette 353 controls how the purge fluid in reservoir 351 is delivered to blood pump 210 and the flow path of the purge fluid from reservoir 351 to blood pump 210. For example, purge cassette 353, as illustrated, includes first purge path diverter 371 and second purge path diverter 372 (e.g., one or more valves) for controlling a pressure and/or flow rate of the purge fluid. Referring again to FIG. 5, purge disc 254 includes one or more measuring device, such as pressure sensors (e.g., a pressure-sensing diaphragm) for measuring purge pressure. Controller 230 is connected to purge cassette 353 and purge disc 254. Purge cassette 353 may contain an actuator that is a rack and pinion that is attached to a piston. Purge disc 254 transmits pressure to controller 230 based on the purge pressure in purge tubing 255. A sensor in controller 230 measures the pressure so that it can be displayed on screen 240. Controller 230 may include a stepper motor. A tic (or step) represents stepper motor positions in units of microsteps, which are also called pulses. In some embodiments, the controller deploys a purge pressure/purge flow curve algorithm along with number of steps/minute of the stepper motor, and pressure measurement by purge disc 254 to calculate the corresponding purge flow rate.

As illustrated, purge tubing 355 provides a fluidic connection passing through purge cassette 353 to blood pump 210. The purge fluid enters blood pump 210 through tubing 373. In some embodiments, a Y-connector and/or a yellow Luer connector are also provided. Y-connector is an adapter that connects purge tubing 255 to blood pump 210. Yellow Luer connector connects purge tubing 255 to a check valve (yellow luer lock) on blood pump 210. Pressure reservoir 257 provides additional filling volume during a purge fluid change. In some embodiments, pressure reservoir 257 includes a flexible rubber diaphragm that provides the additional filling volume by means of an expansion chamber. Infusion filter 258 helps prevent bacterial contamination and air from entering catheter tube 217. Sidearm 259 provides a fluidic connection between infusion filter 258 and plug 270.

During operation, as discussed with respect to FIG. 5 above, controller 230 receives measurements from purge disc 254 and controls the stepper motor's number of ticks to control the purge pressure. In some embodiments, during operation, purge cassette 253 is placed in controller 230 and connected with blood pump 210. As noted above, controller 230 controls and measures purge pressure and calculates purge flow rate via purge cassette 253 and/or purge disc 254. Controller 230 may also control the purge fluid supply. During operation, after exiting purging device 350 through sidearm 259, the purge fluid is channeled through purge lumens (not shown) within catheter tube 217 and plug 270. Sensor cables (not shown) within catheter tube 217, connector cable 260, and plug 270 provide an electrical connection between purge disc 254 and controller 230. Motor cables (not shown) within catheter tube 217, connector cable 260, and plug 270 provide an electrical connection between the motor within motor housing 216 and controller 230. During operation, controller 230 receives measurements from purge disc 254 through the sensor cables and controls the electrical power delivered to the motor within motor housing 216 through the motor cables.

A predetermined threshold purge flow parameter is determined and programmed into controller 230 prior to operation of the blood pump assembly. The predetermined threshold purge flow parameter is such that when this value is met, a remediation protocol is identified. In some embodiments, the predetermined threshold purge flow parameter is set by the blood pump manufacturer. In some embodiments, the predetermined threshold purge flow parameter is set by the user of the blood pump assembly, such as a clinician. In some embodiments, the predetermined threshold purge flow parameter is set by the blood pump manufacturer and the user of the blood pump assembly is prompted to approve or change the value of the predetermined threshold purge flow parameter prior to operation of the blood pump.

The predetermined threshold purge flow parameter may be a predetermined threshold purge flow rate and/or a predetermined threshold purge pressure. In some embodiments, the predetermined threshold purge flow parameter is a predetermined threshold purge flow rate. During operation of the blood pump, normal purge flow rate at the blood pump (i.e., when there is no issue with pump performance due to biological material buildup at the blood pump) ranges from about 2 mL/hour to about 30 mL/hour, depending on the type of blood pump used. For example, in the case of Impella 5.5® (from Abiomed, Inc., Danvers, Mass.), the normal purge flow rate at the blood pump typically ranges from about 2 mL/hour to about 10 mL/hour. However, when there is buildup of biological material at the blood pump, the purge flow rate at the blood pump decreases. In one example, the predetermined threshold purge flow rate is a decrease in the purge flow rate at the blood pump by at least 30% within a 24-hour period. Other examples, including but not limited to, a decrease within a 24-hour period in the purge flow rate at the blood pump by at least 35%, at least 40%, or at least 45% are contemplated. In yet other example, the predetermined threshold purge flow rate at the blood pump is a decrease in the purge flow rate at the blood pump to below 2 mL/hour within a 24-hour period. Other examples, including but not limited to, a decrease in the purge flow rate at the blood pump to below 3 mL/hour within a 6-hour period, an 8-hour period, a 12-hour period, a 16-hour period, an 18-hour period, or a 24-hour period, are contemplated.

In some embodiments, the predetermined threshold purge flow parameter is a predetermined threshold purge pressure. During operation of the blood pump, normal purge pressure at the blood pump (i.e., when there is no issue with pump performance due to biological material buildup at the blood pump) typically ranges from about 300 mmHg to about 1,000 mmHg. However, when there is buildup of biological material at the blood pump, the purge pressure at the blood pump increases. In one example, the predetermined threshold purge pressure is an increase in the purge pressure at the blood pump by at least 30% within a 24-hour period. Other examples, including but not limited to, an increase within a 24-hour period in the purge pressure at the blood pump by at least 35%, at least 40%, or at least 45% are contemplated. In yet other example, the predetermined threshold purge pressure at the blood pump is an increase in the purge pressure at the blood pump to above 700 mmHg within a 24-hour period. Other examples, including but not limited to, an increase in the purge pressure at the blood pump to above 700 mmHg within a 6-hour period, an 8-hour period, a 12-hour period, a 16-hour period, or an 18-hour period, are contemplated.

FIG. 6 shows the direction of flow of the purge fluid through the purging device to the blood pump in the absence of a biological material buildup at the blood pump. As discussed above, controller 230 receives, calculates, and monitors the purge flow parameters from purge disc 254, such as purge pressure and/or purge flow rate. Once controller 230 detects that the purge flow parameter has reached the predetermined threshold purge flow parameter, it identifies a remediation protocol to the blood pump assembly user, such as a clinician. As illustrated in FIG. 8, purge flow alarms may be at flow rates that approach 2 ml/hr or less or exceed 30 ml/hr. If the flow rate exceeds 30 ml/hr, then the pump is rejected. In some embodiments, a display associated with the blood pump 210 (e.g., display 140) may be configured to display the remediation protocol so that a clinician can respond appropriately. In some embodiments, the display may indicate an alert and may optionally include a message such as “Consider tPA application.” As yet another example, the message may be replaced with a different type of information, such as an explanatory statement. For example, a notification may serve as an alert and include a statement describing the cause of the alert. As discussed above, in some embodiments, a monitoring station may use cloud-based technology to securely and remotely display at least some of the recommendations on the associated display (e.g., display 140). For example, a monitoring station may use an online device management system, such as the Impella Connect® from Abiomed, Inc., Danvers, Mass., to securely and remotely display at least some of the recommendations.

Upon receiving the remediation protocol message, the user decides whether to initiate the remediation protocol. In some embodiments, the user may check to see whether there is any kinking in one or more tubing of the blood pump and the purging device that may have caused a buildup in line pressure or biological material that may have resulted in the purge flow parameter reaching the predetermined threshold purge flow parameter. In one embodiment, the user may perform the check prior to initiating the remediation protocol. Upon detecting no kinking in one or more tubing of the blood pump and the purging device, the user may initiate the remediation protocol. For example, the user may press a button on the display (e.g., display 140) that will initiate the remediation protocol.

The remediation protocol helps resolve the purge issue and enables a controlled purge of the blood pump. Controller 230 stops the flow of the purge fluid through purge tubing 355 to blood pump 201 and diverts the flow of the purge fluid to blood pump 210 via supplemental reservoir 380. Controller 230 implements the diversion step by operating first purge path diverter 371 to divert the flow of purge fluid (e.g., dextrose solution) in purge cassette 353 to supplemental purge line 385 and into supplemental reservoir 380, where the purge fluid combines with the supplemental purge additive (e.g., tPA) to form a supplemental purge fluid. The amount of purge fluid passing into supplemental reservoir 380 can be controlled so that the amount of tPA carried to blood pump 201 is modulated. The supplemental purge fluid (which carries, e.g., tPA) flows from supplemental reservoir 380 to blood pump 210 through the supplemental purge fluid supply line 385 via second purge path diverter 372 and tubing 373. In some embodiments, blood pump 210 comprises a motor section and a pump section, and the purge fluid and the supplemental purge fluid are supplied to the motor section. In some embodiments, the supplemental purge fluid is deaerated prior to being supplied to the blood pump. FIG. 7 shows the supplemental purge fluid flow path described above, which is only implemented if the user decides to proceed with the remediation protocol.

In some embodiments, the supplemental purge fluid comprises tPA and dextrose. tPA is preferably lyophilized before it is mixed with the purge fluid. Lyophilized tPA will retain its activity for quite some time, so that purge cassette 353 does not need to be used immediately after manufacture. In some embodiments, dextrose solution passes through the purge fluid supply line 352 to the supplemental purge fluid supply line 385 to the supplemental reservoir 380, where it dissolves lyophilized tPA. For example, 4 mg tPA can be dissolved in 100 mL of 5% dextrose in water. Concentration of tPA in the supplemental purge fluid is from 2 mg/50 mL to 4 mg/50 mL. In some embodiments, the concentration of tPA in the supplemental purge fluid is 2 mg/50 mL. An upper limit to the concentration of the supplemental purge fluid can also be set before the operation of the blood pump. For example, the controller could be programmed to monitor the concentration of the supplemental purge fluid, which is determined by amount of the substance and the volume of the supplemental reservoir. In some embodiments, when the concentration of the supplemental purge fluid reaches a predetermined threshold, the controller could automatically stop the flow of the supplemental purge fluid to the blood pump or identify to the user a remediation of stopping the flow of the supplemental purge fluid to the blood pump.

Just as with the purge fluid, controller 230 receives, calculates, and monitors the supplemental purge flow parameters from purge disc 254, such as pressure and/or flow rate of the supplemental purge fluid at the blood pump. Once controller 230 determines that the supplemental purge flow parameter falls below (in the case of purge flow rate) or exceeds (in the case of purge pressure) the predetermined threshold purge flow parameter, in some embodiments, controller 230 automatically stops the flow of the supplemental purge fluid from supplemental reservoir 380 to blood pump 210 and resumes the flow of the regular purge fluid from purge cassette 353 to blood pump 210 (i.e., purge fluid path shown in FIG. 6 and described above before the remediation protocol was implemented). In some other embodiments, instead of automatically stopping the flow of the supplemental purge fluid to blood pump 210, controller 230 identifies to the user the recommended protocol of initiating the stopping the flow of the supplemental purge fluid to blood pump 210. If the user decides to proceed with the recommendation, controller 230 stops the flow of the supplemental purge fluid to blood pump 210 and resumes the flow of the regular purge fluid from purge cassette 353 to blood pump 210.

Controller 230 resumes the flow of the regular purge fluid from purge cassette 353 to blood pump 210 by operating first purge path diverter 371 and second purge path diverter 372 to restore flow of the purge fluid through purge tubing 355. The direction of flow of the purge fluid to blood pump 210 is as shown in FIG. 6. In some embodiments, the flow of the supplemental purge fluid is stopped and the flow of the regular purge fluid is resumed upon controller 230 determining that the supplemental purge flow parameter at the blood pump exceeds the predetermined threshold purge flow parameter. In some embodiments, flow of the supplemental purge fluid is stopped and the flow of the regular purge fluid is resumed upon controller 230 determining that the supplemental purge flow parameter at the blood pump exceeds the predetermined threshold purge flow parameter by a predetermined amount, for example, at least 30% over a 24-hour period or at least 20% over a 12-hour period.

In other embodiments, the flow of the supplemental purge fluid is stopped and the flow of the purge fluid is resumed upon controller 230 determining that the supplemental purge flow parameter at blood pump 210 has fallen below the predetermined threshold purge flow parameter. In some embodiments, the flow of the supplemental purge fluid is stopped and the flow of the purge fluid is resumed upon controller 230 determining that the supplemental purge flow parameter at blood pump 210 has fallen below the predetermined threshold purge flow parameter by a predetermined amount, for example, at least 30% over a 24-hour period or at least 20% over a 12-hour period. As previously noted, if the flow rate exceeds a threshold of, e.g., 30 ml/hr, the pump is considered to be the cause of the problem and the pump may be replaced.

In some embodiments, the purge fluid comprises aqueous dextrose. In some embodiments, the purge fluid further comprises an anticoagulant, a pH controlling and buffering agent, or combination thereof. In some embodiments, the anticoagulant is warfarin, coumarin, heparin, or a direct thrombin inhibitor. In some embodiments, the anticoagulant is heparin. In some embodiments, the direct thrombin inhibitor is lepirudin, desirudin, argatroban, bivalirudin, or mixtures thereof. In some embodiments, the pH controlling and buffering agent is sodium bicarbonate, sodium citrate, sodium lactate, sodium gluconate, sodium acetate, or sodium pyruvate. In some embodiments, the pH controlling and buffering agent is sodium bicarbonate.

In some embodiments, the purge fluid also comprises no heparin or reduced amounts of heparin. An amount of heparin in the purge fluid is about zero to about 12.5 units per milliliter. In some embodiments, the amount of heparin in the purge fluid is about zero to about 6.25 units per milliliter. In some embodiments, the amount of heparin in the purge fluid is about 1 unit per milliliter to about 6.25 units per milliliter.

As discussed above, in absence of heparin in the purge fluid (e.g., only 5% dextrose or 5% dextrose with DTI, etc.) or in presence of reduced concentration of heparin in the purge fluid, there is an increased chance of buildup of biological material in the motor of the blood pump, which increases the purge pressure and the motor current, while it decreases the purge flow rate, resulting in a mechanical failure of the blood pump. The method and the blood pump assembly of the present disclosure enable a controlled purge recovery by monitoring the purge flow parameters, such as purge flow rate and/or purge pressure, during operation of the blood pump and identifying the remediation protocol to the user when a predetermined threshold purge flow parameter has been met. The administration of supplementary purge fluid, such as purge fluid comprising tPA, will dissolve the buildup of biological material in the motor of the blood pump, thus, resolving the high purge pressure and/or low purge flow rate, so the pump can run as intended. As noted above, the timing of administration of tPA is critical for it to be effective by timely reaching the affected area and in the required concentration. By automating both the monitoring of the conditions when an intervention with tPA may be necessary and the identification of a recommendation to initiate such an intervention gives users, such as clinicians, an opportunity to timely intervene and appropriately mitigate the problem. Additionally, the modification to the purging device disclosed herein provides the users with a quick and easy way to replace the purge fluid with the supplementary purge fluid, such as purge fluid comprising tPA, without undue delay.

In one aspect, described is a method for purging a blood pump, that includes the steps of: i) providing a blood pump in fluid communication with a purging device, wherein the purging device comprises: a purge reservoir configured to be fluidically connected to the blood pump and to receive a purge fluid; and a supplemental reservoir configured to be fluidically connected to the blood pump and to receive a supplemental purge fluid; ii) inserting at least a portion of the blood pump into a patient; iii) operating the blood pump; iv) providing a flow of the purge fluid from the purge reservoir to the blood pump; v) measuring, by a measuring device, a purge flow parameter at the blood pump; and vi) identifying a remediation protocol upon determination, by a controller, that the purge flow parameter at the blood pump meets a predetermined threshold purge flow parameter.

In one aspect, the remediation protocol includes: i) stopping the flow of the purge fluid from the purge reservoir to the blood pump, and ii) starting a flow of the supplemental purge fluid from the supplemental reservoir to the blood pump; iii) measuring, by the measuring device, a supplemental purge flow parameter at the blood pump; and iv) stopping the flow of the supplemental purge fluid from the supplemental reservoir to the blood pump upon determination, by the controller, that the supplemental purge flow parameter at the blood pump has fallen below or exceeds the predetermined threshold purge flow parameter indicating the supplemental purge fluid is no longer required, and resuming the flow of the purge fluid from the purge reservoir to the blood pump.

In any of the above aspects, the flow of the supplemental purge fluid is stopped and the flow of the purge fluid is resumed upon determination, by the controller, that the supplemental purge flow parameter at the blood pump exceeds the predetermined threshold purge flow parameter.

In any of the above aspects, the flow of the supplemental purge fluid is stopped and the flow of the purge fluid is resumed upon determination, by the controller, that the supplemental purge flow parameter at the blood pump exceeds the predetermined threshold purge flow parameter by a predetermined amount, wherein the predetermined amount is at least 30% over a 24-hour period or at least 20% over a 12-hour period.

In any of the above aspects, the purge flow parameter is a purge flow rate and the predetermined threshold purge flow parameter is a decrease in the purge flow rate by a predetermined amount, wherein the predetermined amount is at least 30% within a 24-hour period or at least 20% over a 12-hour period.

In any of the above aspects, the purge flow parameter is a purge flow rate and the predetermined threshold purge flow parameter is a decrease in the purge flow rate to below 3 mL/hour within a 6-hour period.

In any of the above aspects, the purge flow parameter is a purge pressure and the predetermined threshold purge flow parameter is an increase in the purge pressure by a predetermined amount, wherein the predetermined amount is at least 30% within a 24-hour period or at least 20% over a 12-hour period.

In any of the above aspects, the purge flow parameter is a purge pressure and the predetermined threshold purge flow parameter is an increase in the purge pressure to greater than 700 mmHg within a 24-hour period.

In any of the above aspects, the method further comprises deaerating the supplemental purge fluid prior to supplying the supplemental purge fluid to the blood pump.

In any of the above aspects, the method further comprises detecting no kinking in one or more tubing of the blood pump and the purging device.

In any of the above aspects, the purge fluid comprises aqueous dextrose.

In any of the above aspects, the purge fluid further comprises an anticoagulant, a pH controlling and buffering agent, or combination thereof.

In any of the above aspects, the anticoagulant is warfarin, coumarin, heparin, or a direct thrombin inhibitor.

In any of the above aspects, the anticoagulant is heparin.

In any of the above aspects, the direct thrombin inhibitor is lepirudin, desirudin, argatroban, bivalirudin, or mixtures thereof.

In any of the above aspects, the pH controlling and buffering agent is sodium bicarbonate, sodium citrate, sodium lactate, sodium gluconate, sodium acetate, or sodium pyruvate.

In any of the above aspects, the pH controlling and buffering agent is sodium bicarbonate.

In any of the above aspects, the supplemental purge fluid comprises tissue plasminogen activator (tPA).

In any of the above aspects, the supplemental purge fluid further comprises dextrose.

In any of the above aspects, tPA is lyophilized tPA.

In any of the above aspects, concentration of tPA is from 2 mg/50 mL to 4 mg/50 mL.

In any of the above aspects, the blood pump comprises a motor section and a pump section, and wherein the purge fluid and the supplemental purge fluid are supplied to the motor section.

In another aspect, described is a blood pump assembly, comprising: i) a blood pump; ii) a purging device in fluid communication with the blood pump, the purging device comprising: a purge reservoir configured to be fluidically connected to the blood pump and to receive a purge fluid; and a supplemental reservoir configured to be fluidically connected to the blood pump and to receive a supplemental purge fluid; iii) a measuring device configured to measure a purge flow parameter at the blood pump; and iv) a controller configured to: monitor the purge flow parameter at the blood pump; and identify a remediation protocol upon determination that the purge flow parameter at the blood pump meets a predetermined threshold purge flow parameter.

In another aspect, the controller is further configured to implement the remediation protocol by controlling the purging device to: i) stop a flow of the purge fluid from the purge reservoir to the blood pump, and start a flow of the supplemental purge fluid from the supplemental reservoir to the blood pump; ii) measure, by the measuring device, a supplemental purge flow parameter at the blood pump; and iii) stop the flow of the supplemental purge fluid from the supplemental reservoir to the blood pump upon determination that the supplemental purge flow parameter at the blood pump has fallen below or exceeds the predetermined threshold purge flow parameter indicating the supplemental purge fluid is no longer required, and resume the flow of the purge fluid from the purge reservoir to the blood pump.

In any of the above another aspects, the flow of the supplemental purge fluid is stopped and the flow of the purge fluid is resumed upon determination, by the controller, that the supplemental purge flow parameter at the blood pump exceeds the predetermined threshold purge flow parameter.

In any of the above another aspects, the flow of the supplemental purge fluid is stopped and the flow of the purge fluid is resumed upon determination, by the controller, that the supplemental purge flow parameter at the blood pump exceeds the predetermined threshold purge flow parameter by a predetermined amount, wherein the predetermined amount is at least 30% over a 24-hour period or at least 20% over a 12-hour period.

In any of the above another aspects, the purge flow parameter is a purge flow rate and the predetermined threshold purge flow parameter is a decrease in the purge flow parameter by a predetermined amount, wherein the predetermined amount is at least 30% within a 24-hour period or at least 20% over a 12-hour period.

In any of the above another aspects, the purge flow parameter is a purge pressure and the predetermined threshold purge flow parameter is an increase in the purge pressure by a predetermined amount, wherein the predetermined amount is at least 30% within a 24-hour period or at least 20% over a 12-hour period.

In any of the above another aspects, the purging device further comprises: i) a purge fluid supply line fluidically connecting the purge reservoir to the blood pump; and ii) a supplemental purge fluid supply line fluidically connecting the supplemental reservoir to the purge fluid supply line.

In any of the above another aspects, the purge fluid comprises aqueous dextrose.

In any of the above another aspects, the purge fluid further comprises an anticoagulant, a pH controlling and buffering agent, or combination thereof

In any of the above another aspects, the anticoagulant is warfarin, coumarin, heparin, or a direct thrombin inhibitor.

In any of the above another aspects, the anticoagulant is heparin.

In any of the above another aspects, the direct thrombin inhibitor is lepirudin, desirudin, argatroban, bivalirudin, or mixtures thereof.

In any of the above another aspects, the pH controlling and buffering agent is sodium bicarbonate, sodium citrate, sodium lactate, sodium gluconate, sodium acetate, or sodium pyruvate.

In any of the above another aspects, the pH controlling and buffering agent is sodium bicarbonate.

In any of the above another aspects, the supplemental purge fluid comprises tissue plasminogen activator (tPA).

In any of the above another aspects, the supplemental purge fluid further comprises dextrose.

In any of the above another aspects, the tPA is lyophilized tPA.

In any of the above another aspects, concentration of tPA is from 2 mg/50 mL to 4 mg/50 mL.

In any of the above another aspects, the blood pump comprises a motor section and a pump section, and wherein the purge fluid and the supplemental purge fluid are supplied to the motor section.

In this specification, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

While particular embodiments of this technology have been described, it will be evident to those skilled in the art that the present technology may be embodied in other specific forms without departing from the essential characteristics thereof. The present embodiments and examples are therefore to be considered in all respects as illustrative and not restrictive. It will further be understood that any reference herein to subject matter known in the field does not, unless the contrary indication appears, constitute an admission that such subject matter is commonly known by those skilled in the art to which the present technology relates.

1. A method for purging a blood pump, the method comprising:
providing a blood pump in fluid communication with a purging device, wherein the purging device comprises:
a purge reservoir configured to be fluidically connected to the blood pump and to receive a purge fluid; and
a supplemental reservoir configured to be fluidically connected to the blood pump and to receive a supplemental purge fluid;
inserting at least a portion of the blood pump into a patient;
operating the blood pump;
providing a flow of the purge fluid from the purge reservoir to the blood pump;
measuring, by a measuring device, a purge flow parameter at the blood pump; and
identifying a remediation protocol upon determination, by a controller, that the purge flow parameter at the blood pump meets a predetermined threshold purge flow parameter.

2. The method of claim 1, wherein the remediation protocol comprises:
stopping the flow of the purge fluid from the purge reservoir to the blood pump, and starting a flow of the supplemental purge fluid from the supplemental reservoir to the blood pump;
measuring, by the measuring device, a supplemental purge flow parameter at the blood pump; and
stopping the flow of the supplemental purge fluid from the supplemental reservoir to the blood pump upon determination, by the controller, that the supplemental purge flow parameter at the blood pump has fallen below or exceeds the predetermined threshold purge flow parameter indicating the supplemental purge fluid is no longer required, and resuming the flow of the purge fluid from the purge reservoir to the blood pump.

3. The method of claim 1, wherein the flow of the supplemental purge fluid is stopped and the flow of the purge fluid is resumed upon determination, by the controller, that the supplemental purge flow parameter at the blood pump exceeds the predetermined threshold purge flow parameter.

4. The method of claim 1, wherein the flow of the supplemental purge fluid is stopped and the flow of the purge fluid is resumed upon determination, by the controller, that the supplemental purge flow parameter at the blood pump exceeds the predetermined threshold purge flow parameter by a predetermined amount, wherein the predetermined amount is at least 30% over a 24-hour period or at least 20% over a 12-hour period.

5. The method of claim 1, wherein the purge flow parameter is a purge flow rate and the predetermined threshold purge flow parameter is a decrease in the purge flow rate by a predetermined amount, wherein the predetermined amount is at least 30% within a 24-hour period or at least 20% over a 12-hour period.

6. The method of claim 1, wherein the purge flow parameter is a purge flow rate and the predetermined threshold purge flow parameter is a decrease in the purge flow rate to below 3 mL/hour within a 6-hour period.

7. The method of claim 1, wherein the purge flow parameter is a purge pressure and the predetermined threshold purge flow parameter is an increase in the purge pressure by a predetermined amount, wherein the predetermined amount is at least 30% within a 24-hour period or at least 20% over a 12-hour period.

8. The method of claim 1, wherein the purge flow parameter is a purge pressure and the predetermined threshold purge flow parameter is an increase in the purge pressure to greater than 700 mmHg within a 24-hour period.

9. The method of claim 2, further comprising deaerating the supplemental purge fluid prior to supplying the supplemental purge fluid to the blood pump.

10. The method of claim 1, further comprising detecting no kinking in one or more tubing of the blood pump and the purging device.

11. The method of claim 1, wherein the purge fluid comprises aqueous dextrose.

12. The method of claim 11, wherein the purge fluid further comprises an anticoagulant, a pH controlling and buffering agent, or combination thereof.

13. The method of claim 12, wherein the anticoagulant is warfarin, coumarin, heparin, or a direct thrombin inhibitor.

14. The method of claim 13, wherein the anticoagulant is heparin.

15. The method of claim 13, wherein the direct thrombin inhibitor is lepirudin, desirudin, argatroban, bivalirudin, or mixtures thereof.

16. The method of claim 12, wherein the pH controlling and buffering agent is sodium bicarbonate, sodium citrate, sodium lactate, sodium gluconate, sodium acetate, or sodium pyruvate.

17. The method of claim 16, wherein the pH controlling and buffering agent is sodium bicarbonate.

18. The method of claim 1, wherein the supplemental purge fluid comprises tissue plasminogen activator (tPA).

19. The method of claim 18, wherein the supplemental purge fluid further comprises dextrose.

20. The method of claim 18, wherein tPA is lyophilized tPA.

21. The method of claim 18, wherein concentration of tPA is from 2 mg/50 mL to 4 mg/50 mL.

22. The method of claim 1, wherein the blood pump comprises a motor section and a pump section, and wherein the purge fluid and the supplemental purge fluid are supplied to the motor section.

23. A blood pump assembly, comprising:
a blood pump;
a purging device in fluid communication with the blood pump, the purging device comprising:
a purge reservoir configured to be fluidically connected to the blood pump and to receive a purge fluid; and
a supplemental reservoir configured to be fluidically connected to the blood pump and to receive a supplemental purge fluid;
a measuring device configured to measure a purge flow parameter at the blood pump; and
a controller configured to:
monitor the purge flow parameter at the blood pump; and
identify a remediation protocol upon determination that the purge flow parameter at the blood pump meets a predetermined threshold purge flow parameter.

24. The blood pump assembly of claim 23, wherein the controller is further configured to implement the remediation protocol by controlling the purging device to:
stop a flow of the purge fluid from the purge reservoir to the blood pump, and start a flow of the supplemental purge fluid from the supplemental reservoir to the blood pump;
measure, by the measuring device, a supplemental purge flow parameter at the blood pump; and
stop the flow of the supplemental purge fluid from the supplemental reservoir to the blood pump upon determination that the supplemental purge flow parameter at the blood pump has fallen below or exceeds the predetermined threshold purge flow parameter indicating the supplemental purge fluid is no longer required, and resume the flow of the purge fluid from the purge reservoir to the blood pump.

25. The blood pump assembly of claim 24, wherein the flow of the supplemental purge fluid is stopped and the flow of the purge fluid is resumed upon determination, by the controller, that the supplemental purge flow parameter at the blood pump exceeds the predetermined threshold purge flow parameter.

26. The blood pump assembly of claim 24, wherein the flow of the supplemental purge fluid is stopped and the flow of the purge fluid is resumed upon determination, by the controller, that the supplemental purge flow parameter at the blood pump exceeds the predetermined threshold purge flow parameter by a predetermined amount, wherein the predetermined amount is at least 30% over a 24-hour period or at least 20% over a 12-hour period.

27. The blood pump assembly of claim 23, wherein the purge flow parameter is a purge flow rate and the predetermined threshold purge flow parameter is a decrease in the purge flow parameter by a predetermined amount, wherein the predetermined amount is at least 30% within a 24-hour period or at least 20% over a 12-hour period.

28. The blood pump assembly of claim 23, wherein the purge flow parameter is a purge pressure and the predetermined threshold purge flow parameter is an increase in the purge pressure by a predetermined amount, wherein the predetermined amount is at least 30% within a 24-hour period or at least 20% over a 12-hour period.

29. The blood pump assembly of claim 23, wherein the purging device further comprises:
a purge fluid supply line fluidically connecting the purge reservoir to the blood pump; and
a supplemental purge fluid supply line fluidically connecting the supplemental reservoir to the purge fluid supply line.

30. The blood pump assembly of claim 23, wherein the purge fluid comprises aqueous dextrose.

31. The blood pump assembly of claim 30, wherein the purge fluid further comprises an anticoagulant, a pH controlling and buffering agent, or combination thereof.

32. The blood pump assembly of claim 31, wherein the anticoagulant is warfarin, coumarin, heparin, or a direct thrombin inhibitor.

33. The blood pump assembly of claim 32, wherein the anticoagulant is heparin.

34. The blood pump assembly of claim 32, wherein the direct thrombin inhibitor is lepirudin, desirudin, argatroban, bivalirudin, or mixtures thereof.

35. The blood pump assembly of claim 31, wherein the pH controlling and buffering agent is sodium bicarbonate, sodium citrate, sodium lactate, sodium gluconate, sodium acetate, or sodium pyruvate.

36. The blood pump assembly of claim 35, wherein the pH controlling and buffering agent is sodium bicarbonate.

37. The blood pump assembly of claim 23, wherein the supplemental purge fluid comprises tissue plasminogen activator (tPA).

38. The blood pump assembly of claim 37, wherein the supplemental purge fluid further comprises dextrose.

39. The blood pump assembly of claim 37, wherein the tPA is lyophilized tPA.

40. The blood pump assembly of claim 37, wherein concentration of tPA is from 2 mg/50 mL to 4 mg/50 mL.

41. The blood pump assembly of claim 23, wherein the blood pump comprises a motor section and a pump section, and wherein the purge fluid and the supplemental purge fluid are supplied to the motor section.
